(19) United States
(12) Patent Application Publication
(10) Pub. No.: US 2006/0118107 A1
(43) Pub. Date: Jun. 8, 2006

(54) Tubular nozzles for use in systems for delivering medicaments

(76) Inventor: Michael L. King, Durham, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/533,206

(22) PCT Filed: Oct. 30, 2003
(86) PCT No.: PCT/US03/34536

§ 371 Date: Apr. 28, 2005

Related U.S. Application Data
(60) Provisional application No. 60/422,203, filed on Oct. 30, 2002.

(51) Int. Cl. A61M 11/00

(52) U.S. Cl. 128/200.23; 128/200.14; 128/200.19

(57) ABSTRACT

A system for delivering a pharmaceutical formulation to a patient that includes a container having a pharmaceutical formulation including at least one medicament present therein, a metering assembly in communication with the container, and a tubular nozzle having an inlet configured in size to communicate with the metering assembly, and an outlet for directing the medicament to a patient is disclosed. The tubular nozzle preferably has a defined length and a longitudinal axis that is curvilinear through the defined length of the tubular nozzle.

### CROSS-REFERENCE TO RELATED APPLICATIONS

The present application claims priority to provisional application No. 60/422,203 filed Oct. 30, 2002, the disclosure of which is incorporated herein by reference in its entirety.

### FIELD OF THE INVENTION

The present invention generally relates to systems for use in delivering medicaments to patients, and methods of using the same.

### BACKGROUND OF THE INVENTION

A variety of systems for delivering medicaments in a fluid medium are widely known in the art. Examples include aerosol systems which typically deliver one or more medicaments in combination with a propellant, as well as liquid systems that employ a pump. Such systems may be administered in a number of ways including, for example, orally and intranasally.

With respect to oral inhalers, medicaments, broadly including therapeutic, prophylactic and diagnostic agents, may be delivered locally to the lung or systemically through the lung for the treatment, prophylaxis or diagnosis of illnesses and other conditions. Many devices are used to deliver medicaments to the lung, including, as an example, metered dose inhalers (MDIs). MDIs are aerosol delivery systems having a reservoir of compressed, low boiling point liquid propellant formulated with a medicament. MDIs are designed to meter a predetermined quantity of the medicament formulation and to dispense the dose as an inhalable particulate cloud, or plume.

In one example of a conventional MDI, the patient typically orients the inhaler so that the canister is substantially vertical with the valve down (which orients the mouthpiece substantially horizontally), then seals their lips over the mouthpiece and actuates the MDI by depressing the canister into the actuator. Upon actuation, a metered dose is released by the valve and expands into and through the internal expansion chamber. The pressure of the rapidly expanding and evaporating propellant forces the metered dose through the nozzle channel, atomizing the liquid portion of the dose into small droplets. These droplets along with propellant vapor and the drug particles (which may be contained in the droplets) form a plume of aerosolized drug. The patient breathes in through the mouthpiece as the plume is dispensed and inhales the drug dose as it exits the inhaler.

It is appreciated by those skilled in the art that medicaments delivered through inhalation devices are intended to optimally target specific sites in the pulmonary system. These sites include the nasal passages, the throat, and various locations within the lung, such as the bronchi, bronchioles and alveolar regions. The ability to deliver drugs to a target area is often largely dependent on the aerodynamic size of the medicament particles. As currently believed to be understood, particles having an aerodynamic diameter of less than 2 microns are considered to be potentially optimal for deposition in the alveolar region of the lung. Particles that have an aerodynamic diameter of between 2 and approximately 5 microns may be more suitable for delivery to the bronchiole or bronchi regions. Particles with an aerodynamic size range greater than 6 microns, and more preferably 10 microns, are typically suitable for delivery to the laryngeal region, throat or nasal passages.

As used herein, particles of six microns or less are referred to as “respirable” or “within the respirable range.” In turn, the percentage of the particles within a given dose of aerosolized medicament that is of “respirable” size, as compared to the total dose, is referred to as the “respirable fraction,” “fine particle mass” (FPM) of the dose. For the purposes of this discussion, particles may consist of propellant droplets containing drug, individual drug particles, agglomerated drug particles, or a combination of these.

It is desirable to achieve a high device delivery efficiency. It is believed that the delivery efficiency of the device is often determined by various factors such as, for example, the size and velocity of the particles or droplets generated, and the size and velocity of the plume. Achieving maximum efficiency is advantageous in order to provide the desired therapeutic benefit while minimizing side effects.

In general, FPM has been shown to increase with decreasing diameter of the actuator nozzle channel. Also, generally, plume velocity decreases with decreasing nozzle diameter. This relationship is believed to be relevant since a high plume velocity tends to cause more droplets/particles to impact the back of the throat, resulting in increased oropharyngeal drug deposition. Thus, respirable fraction should be maximized and plume velocity should be sufficiently low to attempt to maximize the amount of medicament that reaches the lung.

However, small diameter nozzles may be perceived as presenting various potential disadvantages. A small spray orifice may increase the duration of the plume, which could lead to inconsistent drug delivery with current-art valves. A small orifice is capable of restricting flow, which often causes increased deposition of drug on the surfaces of the expansion chamber or nozzle channel of conventional devices. In turn, surface buildup or detachment of aggregated drug deposits may potentially cause clogging of the nozzle, reducing or blocking the dose delivered to the patient. A small orifice is also capable of increasing the spray angle of the plume, making the plume more disperse, and thus potentially increasing deposition of drug on the interior surfaces of the device (e.g., the mouthpiece, the nozzle block) and on the inside of the mouth. This unintended deposition tends to reduce the amount of drug delivered to the intended target (the lung), and increases the amount of drug that may be ingested and therefore potentially contribute to causing side effects. In current actuators, which are injection molded in plastic, a smaller orifice may be more difficult to manufacture accurately.

The respirable fraction of the aerosol plume emitted from a conventional MDI may be increased by the use of an add-on (called a “spacer”) attached to the MDI mouthpiece. A spacer is potentially capable of effectively slow down the MDI aerosol plume, thus allowing more time for the aerosol droplets to evaporate before inhalation. The patient accordingly may inhale a dryer, warmer and softer aerosol plume. As a result, less drug deposition in the mouth and throat is capable of occurring. Spacers also allow greater flexibility in synchronizing triggering the MDI and patient inhalation.

Notwithstanding the above, drug loss in a spacer may be sizeable because of impaction deposition and gravitational settling inside the spacer. The dose leaving the spacer tends to be smaller than the dose emitted from an MDI without a spacer; The net effect is that a spacer may shift the drug deposition from patient's mouth and throat to the spacer. While spacers may be useful in terms of percentage of respirable particles exiting the spacer, they disadvantageously fail to significantly increase the amount of respirable material entering the lung.

Conventional nozzles and MDI's thereof tend to incorporate narrow diameter orifices in an attempt to improve fine particle mass. However, the spray plume is often more dispersed, which may result in increased drug particle deposition in the nozzle, mouth and throat. As discussed herein, other conventional devices incorporate spacers which also intended to increase drug particle deposition. Nonetheless, the use of such spacers is often problematic in that excessive drug deposition may occur within the structure of the spacer.

In view of the above, there is a need in the art to provide for a medicament delivery system that allows for a potential increase in FPM while minimizing excessive medicament deposition within the system and within the patient's oropharynx. There is a need for such a system for use in a wide number of applications including, without limitation, those encompassing oral and intranasal inhalation.

### SUMMARY OF THE INVENTION

In one aspect, the invention provides a system for delivering at least one medicament to a patient. The system comprises a container having a pharmaceutical formulation comprising at least one medicament present therein; a metering assembly in communication with the container; a tubular nozzle having an inlet configured in size to communicate with the metering assembly, and an outlet for directing the medicament to a patient. The tubular nozzle has a defined length and a longitudinal axis that is curvilinear throughout the defined length of the tubular nozzle. In particular, the tubular nozzle has a radius of curvature at least 4 times the inner diameter of the tubular nozzle present within the curved portion.

In another aspect, the invention provides a method of administering at least one medicament to a patient. The method comprises providing a system as defined herein and activating the system to deliver the at least one medicament to the patient.

These and other aspects are provided by the present invention as set forth herein.

### BRIEF DESCRIPTION OF THE DRAWINGS

FIG. 1 is a diagram of a tubular nozzle in accordance with the present invention.

FIG. 2 is a cross-sectional view of a medicament delivery system employing a tubular nozzle according to the present invention.

FIG. 3 is a cross-sectional view of a medicament delivery system employing a tubular nozzle according to the present invention.

FIGS. 4 through 8 illustrate side views of various tubular nozzles according to the present invention.

FIGS. 9 through 19 illustrate side views of various tubular nozzle inlets in communication with dispensing passages in accordance with the present invention.

FIGS. 20 through 25 illustrate various medicament canisters employing tubular nozzles attached thereto according to the present invention.

FIG. 26 is a cross-sectional view of a medicament delivery system according to the present invention.

### DETAILED DESCRIPTION OF THE INVENTION

The invention will now be described with respect to the embodiments set forth herein, including, but not limited to, those described by the drawings. It should be appreciated that these embodiments are set forth to illustrate the invention, however, the invention is not limited to these embodiments.

All publications, patents, and patent applications cited herein, whether supra or infra, are hereby incorporated herein by reference in their entirety to the same extent as if each individual publication, patent, or patent application was specifically and individually indicated to be incorporated by reference.

It must be noted that, as used in the specification and appended claims, the singular forms “a”, “an” and “the” include plural referents unless the content clearly dictates otherwise.

In one aspect, the invention provides a system for delivering at least one medicament to a patient. The system comprises a container having a pharmaceutical formulation comprising at least one medicament present therein; a metering assembly in communication with the container; and a tubular nozzle having an inlet configured in size to communicate with the metering assembly, and an outlet for directing the medicament to a patient. The tubular nozzle has a defined length and a longitudinal axis that is curvilinear throughout the defined length of the tubular nozzle.

In a particular embodiment, the system is present as an oral inhaler. In such an embodiment, the container is present as a canister capable of withstanding pressure, the pharmaceutical formulation is present as a pharmaceutical aerosol formulation comprising the at least one medicament and at least one propellant, the metering assembly is present as a metering valve assembly including a valve stem, wherein a passage for dispensing the at least one medicament is positioned in the valve stem; and wherein the inlet of the tubular nozzle is configured in size to communicate with the dispensing passage.

In one embodiment, the inhaler further comprises a means of actuation to dispense a metered dose of medicament assisting in delivering medicament to a patient, and a means of allowing a patient to inhale the medicament, e.g., a mouthpiece.

In one embodiment, the inlet of the tubular nozzle is oriented substantially in alignment or in alignment with the dispensing passage.

In one embodiment, the diameter of the tubular nozzle is substantially similar to the diameter of the dispensing passage.

The term “curvilinear” refers to the nozzle being composed of curved and/or straight line segments, where all transitions between segments are tangent at their ends, i.e., free or substantially free from angularity, sharp turns, corners, or other discontinuities. Furthermore, the channel formed by the inner walls of the tubular nozzle is substantially free of corners, discontinuities, and surfaces perpendicular to the flow direction along the entire length of the tubular nozzle. Moreover, fluid flow throughout the tubular nozzle is unobstructed. The flow direction of the tubular nozzle is defined as being parallel or substantially parallel to the central axis, in the direction from the inlet toward the outlet.

The tubular nozzle may be configured in a variety of structures in accordance with the present invention. Various embodiments of features of a tubular nozzle will now be described. In particular, these embodiments may be combined to arrive at the design of a particular tubular nozzle. Any particular feature described may or may not be incorporated in a given tubular nozzle. The design of the tubular nozzle is not limited to only those embodiments described herein.

As set forth herein, the tubular nozzle has an inlet at the proximal end configured in size to communicate with the outlet of the dispensing passage positioned in the valve stem. In one embodiment, the inner diameter of the tubular nozzle inlet is the same or substantially the same as the diameter of the dispensing passage outlet.

As set forth herein, the tubular nozzle includes an outlet located at the distal end. At the end of the outlet is the exit orifice. The dispensed dose emanates from the exit orifice, typically in the form of a spray, mist, plume, or cloud to be directed toward the patient's oropharynx for inhalation. As such, it is believed that the outlet design influences the flow transition of the dispensed dose from within the tubular nozzle to the external surroundings of the tubular nozzle. In one embodiment, the outlet is of constant diameter. In another embodiment, the outlet is of increasing or decreasing diameter. In another embodiment, the outlet diameter is the same or substantially the same as the throat diameter. In another embodiment, the end of the outlet protrudes or is recessed from the end of the inhaler mouthpiece. In a particular embodiment, the end of the outlet is located over a range from being substantially even with the end of the mouthpiece to being recessed from less than to about 25 mm from the end of the mouthpiece.

In various embodiments, the tubular nozzle includes a throat. A throat is defined as the portion of the tubular nozzle of smallest diameter. The throat may be positioned near the inlet, at or near the outlet, or anywhere between the inlet and outlet of the tubular nozzle. Although not intending to be bound by, theory, as the smallest-diameter portion of the tubular nozzle, the throat is believed to largely influence the rate of release and atomization/aerosolization of a dispensed dose of medicament. In a particular embodiment, the throat is coaxial with the tubular nozzle axis.

In various embodiments, the tubular nozzle of the invention may include one or more tapered sections. A “tapered section” is defined as a length of the tubular nozzle over which the diameter changes, connecting the diameters at each end of the tapered portion via a smooth fluid flow path. The tapered sections represent the diameter transitions within the tubular nozzle, and are designed to maintain a smooth fluid flow path. The tapered section(s) may be configured such that the tubular nozzle undergoes a decrease in diameter along the nozzle length in the direction moving toward the distal end of the nozzle. Conversely, the tapered section(s) may be configured such that the tubular nozzle undergoes an increase in diameter along the nozzle length in the direction moving toward the distal end of the nozzle. The tapered section(s) may be located at any position along the tubular nozzle. As an example, the tapered section(s) may be located at the proximal end of the tubular nozzle (e.g., inlet), the distal end of the tubular nozzle (e.g., outlet), and/or one or more locations between the proximal and distal ends. Multiple tapered sections may be present in the tubular nozzle. In accordance with the invention, the tapered section provides a smooth reduction in diameter with no sharp edges and corners. In a particular embodiment, the tapered section(s) has an angle θ of less than 45°, more preferably less than 30°, yet more preferably less than 15°.

In another embodiment, the tubular nozzle includes at least one linear portion. For the purposes of the invention, the term “linear portion” is defined to encompass those portion(s) of the tubular nozzle that are straight, i.e., without curvature. The linear portion(s) may be located at various locations of the tubular nozzle. As an example, in one embodiment, the linear portion(s) can be located at a proximal end of the tubular nozzle relative to the valve stem. In another embodiment, the linear portion(s) can be located at a distal end of the tubular nozzle relative to the valve stem. In one embodiment, the tubular nozzle may include a plurality of linear portions located at the distal end and/or the proximal end of the tubular nozzle, as well as at locations along the tubular nozzle between the proximal end and the distal end.

In one embodiment, for example, the tubular nozzle may include one or more curved portions. For the purposes of the invention, a curved portion is one which possesses a radius of curvature throughout its length. The curved portion(s) allows the tubular nozzle to be routed as desired in the inhaler device while maintaining a smooth fluid flow path, and may have various dimensions. In one embodiment, the curved portion(s) have a radius of curvature of at least about 2.5, 4, or 5 times the inner diameter of the tubular nozzle within the curved portion. In a particular embodiment, the radius of curvature may range up to about 10 times the inner diameter of the tubular nozzle within the curved portion. It is believed that a radius of curvature of less than 2.5 is disadvantageous in that the tubular nozzle too closely approaches a sharp angle such that the nozzle may not be curvilinear. Advantageously, it is believed that the radius of curvature of the invention assists in providing a smooth flow path in the tubular nozzle.

In one embodiment, the tubular nozzle may include one or more coils to aid in packaging the tubular nozzle in an inhaler device. With the tubular nozzle properly coiled, the inhaler device may be made compact even when using a very long tubular nozzle. The number, size, spacing, and orientation of the coils may be chosen to fit the desired tubular nozzle length within the desired inhaler device configuration.

In another embodiment, a means of connection is present at the proximal end of the tubular nozzle to substantially sealably connect the tubular nozzle to the valve stem. Such means of connection includes various suitable connectors including, without limitation, those described herein. In a particular embodiment, the diameter of a suitable connector is similar in size to the diameter of the valve stem or dispensing passage. The tubular nozzle and connector may be of one-piece construction such that they are made from one piece of material. Alternatively, the tubular nozzle and the connector may be of two-piece construction such that they are made as two separate pieces that are subsequently assembled.

In a particular embodiment, the inhaler of the present invention includes a mouthpiece. In one embodiment, a portion of the tubular nozzle containing the tubular nozzle outlet is positioned in the mouthpiece. In another embodiment, the outlet of the tubular nozzle is in alignment or substantial alignment with the outlet of the mouthpiece. In another embodiment, the outlet of the tubular nozzle is coaxial with the outlet and/or central axis of the mouthpiece. In another embodiment, the outlet is at an angle relative to the outlet of the mouthpiece.

In various embodiments, the tubular nozzle may be configured so as to encompass a number of design parameters. As an example, in a particular embodiment, the tubular nozzle may have a preferred overall length ranging at a lower end from about 4, 50, 100, or 250, or 400 mm to about 600, 750, 900, or 1000 mm at an upper end. In one embodiment, the tubular nozzle may have a particular inner, e.g., throat, diameter ranging from about 0.1 mm to about 0.5 mm. In a particular embodiment, the tubular nozzle may have a diameter of the channel between the inlet and the throat, or between the throat and the outlet, ranging from about 1, 2, or 4 to 6, 8, or 10 times the throat diameter. Notwithstanding the above, it should be appreciated that embodiments other than those set forth above are encompassed without departing from the scope of the present invention.

For the purposes of the invention, the tubular nozzle may be configured in a number of geometries. As an example, in various embodiments, the tubular nozzle may contain cross-sections selected from a wide range of choices such as, without limitation, circular, oval, square, rectangular polygonal, and the like.

In a particular embodiment, the inhaler includes a connector that receives a proximal end of the tubular nozzle, the connector engaging the valve stem. Preferably, the tubular nozzle is entirely external to the valve stem, although, as set forth herein, other embodiments are contemplated. In various embodiments, the tubular nozzle and the connector may be constructed from identical or similar materials. In other embodiments, the tubular nozzle and the connector may be constructed from different materials. A number of various materials can be used in constructing the tubular nozzle and receptacle. Examples of materials includes metallic materials, such as, without limitation, stainless steel (e.g., 316L Medical Grade), gold, iron, nickel, copper, titanium, tantalum, ferrous, brass and aluminum, as well as combinations thereof such as, for example, alloys. The tubular nozzle and/or connector may also be made from a polymeric material. Exemplary polymeric materials include, without limitation, polyethyhlene (PE), polypropylene (PP), polymethylmethylacrylate (PMMA), polyvinyl chloride (PVC), polyvinyidiene chloride (PVDC), polyvinyl fluoride (PVF), polyvinyldiene fluoride (PVDF), polychlorotrifluoroethylene (PCTFE), polytetrafluoroethylene (PTFE), fluorinated ethylene propylene (FEP), perfluroroalkoxy (PFA), polyamide (PA), polyethylene terephthalate (PET), polybutylene terephthalate (PBT), polyetherimide (PEI), polyamideimide (PAI), polyimide (PI), polysulfone (PS), polyarylsulfone (PAS) polyethersulfone (PES), polyphenylene sulfide (PPS), polyetheretherketone (PEEK), polydimethylsiloxane (PDMS) and polycarbonate (PC). Combinations (e.g., blends) thereof are also encompassed. These polymeric materials are generally available from typical suppliers such as DuPont, Dow, General Electric, ICI, 3M, Monsanto, Amoco, BASF, Allied Signal, Bayer, Eastman, Phillips, LNP, and the like. In various embodiments, fabrication materials may also be of composite structure, comprising a base substrate and layer coating the substrate. The base substrate material may comprise any of the aforementioned materials, or any other material known to the art which would be deemed suitable for the purposes contemplated herein. The coating layer may include a fluoropolymer, silicone or fluorosilicone based material or other material or material blend with low adhesion properties, or which is smooth, or which posses low surface energy.

The materials may be a pure material or may be a blended material, such as blended polymeric materials. Alternatively, multiple layers of coating materials may be sequentially applied onto the base material.

The performance of the nozzle and connection means may be further enhanced by employing a coating having a low surface energy. Smooth surfaces may also be employed.

Typical low surface energy materials include fluoropolymer and silicone materials such as, for example, PTFE, FEP, PFA, PVDF and PDMS. The material may itself posses such properties, or may be coated to possess such properties. Plasma coating may be used to impart fluoropolymers, silicones, or other low surface energy coatings to the material so treated.

The above coatings can be applied through any coating/manufacturing process known in the industry, including spray coating, dip coating, electrostatic coating, chemical vapor deposition, plasma enhanced chemical vapor deposition, cold plasma deposition and laminating.

The tubular nozzle, connector, and inhaler employing the same may be manufactured according to various techniques, including those known in the art. For example, with respect to embodiments that employ metallic materials for the tubular nozzle or connector, tube fabrication processes such as cutting, drawing, reducing/expanding, flaring, rounding, coining, bending, and coiling can be used, or other metalworking processes such as machining, broaching, grinding, stamping, drawing, bending, metal injection molding, casting, and powder metallurgy can be used. In embodiments wherein the tubular nozzle or connector is formed from polymeric materials, injection molding, extrusion, or thermoforming can be used. For embodiments in which the tubular nozzle and connector are of two-piece construction, various assembly processes may be used, including snap fit, interference fit, shrink fit, brazing, soldering, welding, adhesive bonding, insert molding, and two-shot injection molding. Moreover, a number of methods can be employed to assemble the tubular nozzle to an inhaler. Examples of such methods include, without limitation, snap fit, interference fit, adhesive bonding, insert molding, two-shot injection molding, capture by additional component(s), and clamping by additional component(s).

The oral inhaler (e.g., MDI) employing the tubular nozzle of the present invention may operated in various and accepted manners. For example, in one embodiment, the canister is depressed into the actuator. The motion of the canister causes the metering valve to meter a fixed volume of the fluid forming an individual dose. The metered dose of the fluid passes into and through the valve stem and into the tubular nozzle. Upon leaving the pressurized environment of the canister and metering chamber, the propellant component of the fluid expands within the tubular nozzle. The aerosolized and metered pharmaceutical formulation (i.e., the dose) is expelled from the nozzle exit orifice as droplets or particles (i.e., the plume). The patient breathes in through the mouthpiece of the inhaler as the dose is dispensed and inhales the drug plume as it exits the inhaler. It should be appreciated that the mouthpiece may be oriented at various angles relative to the mouth of the patient.

Medicaments, which may be administered in the aerosol formulations, include a variety of drugs, such as, for example, those which are useful in inhalation therapy. Combinations of medicaments may also be employed. Appropriate medicaments may thus be selected from, for example, analgesics, (e.g., codeine, dihydromorphine, ergotamine, fentanyl or morphine); anginal preparations, (e.g., diltiazem; antiallergics, e.g., cromoglycate, ketotifen or nedocromil); antiinfectives (e.g., cephalosporins, penicillins, streptomycin, sulphonamides, tetracyclines and pentamidine); antihistamines, (e.g., methapyrilene); anti-inflammatories, (e.g., beclomethasone dipropionate, fluticasone propionate, flunisolide, budesonide, rofleponide, mometasone furoate, ciclesonide, triamcinolone acetonide or 6α, 9α-difluoro-11β-hydroxy-16α-methyl-3-oxo-17α-propionyloxy-androsta-1,4-diene-17β-carbothioic acid S-(2-oxo-tetrahydro-furan-3-yl) ester)); antitussives, (e.g., noscapine; bronchodilators, e.g., albuterol (e.g. as sulphate), salmeterol (e.g. as xinafoate), ephedrine, adrenaline, fenoterol (e.g., as hydrobromide), formoterol (e.g., as fumarate), isoprenaline, metaproterenol, phenylephrine, phenylpropanolamine, pirbuterol (e.g., as acetate), reproterol (e.g., as hydrochloride), rimiterol, terbutaline (e.g., as sulphate), isoetharine, tulobuterol or 4-hydroxy-7-[2-[[2-[[3-(2-phenylethoxy)propyl]sulfonyl]ethyl]amino]ethyl-2(3H)-benzothiazolone); diuretics, (e.g., amiloride; anticholinergics, e.g., ipratropium (e.g., as bromide), tiotropium, atropine or oxitropium); hormones, (e.g., cortisone, hydrocortisone or prednisolone); xanthines, (e.g., aminophylline, choline theophyllinate, lysine theophyllinate or theophylline); therapeutic proteins and peptides, (e.g., insulin). It will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimise the activity and/or stability of the medicament. It will be further clear to a person skilled in the art that where appropriate, the medicaments may be used in the form of a pure isomer, for is example, R-salbutamol or RR-formoterol.

Particular medicaments for administration using pharmaceutical aerosol formulations in accordance with the invention include anti-allergics, bronchodilators and anti-inflammatory steroids of use in the treatment of respiratory disorders such as asthma by inhalation therapy, for example cromoglycate (e.g. as the sodium salt), salbutamol (e.g. as the free base or the sulphate salt), salmeterol (e.g. as the xinafoate salt), formoterol (e.g. as the fumarate salt), terbutaline (e.g. as the sulphate salt), reproterol (e.g. as the hydrochloride salt), a beclomethasone ester (e.g. the dipropionate), a fluticasone ester (e.g. the propionate). Medicaments useful in erectile dysfunction treatment (e.g., PDE-V inhibitors such as vardenafil HCl, along with alprostadil and sildenafil citrate) may also be employed.

Salmeterol, especially salmeterol xinafoate, salbutamol, fluticasone propionate, beclomethasone dipropionate and physiologically acceptable salts and solvates thereof are especially preferred.

It will be appreciated by those skilled in the art that the formulations according to the invention may, if desired, contain a combination of two or more active ingredients. Formulations containing two active ingredients are known for the treatment of respiratory disorders such as asthma and COPD, for example, formoterol (e.g. as the fumarate) and budesonide, salmeterol (e.g. as the xinafoate salt) and fluticasone (e.g. as the propionate ester), salbutamol (e.g. as free base or sulphate salt) and beclomethasone (as the dipropionate ester) are preferred.

In one embodiment, a particular combination is a combination of fluticasone propionate and salmeterol, or a salt thereof (particularly the xinafoate salt). It should be understood that the medicaments that may be used in conjunction with the inhaler are not limited to those described herein.

In appropriate instances, particular embodiments of formulations for use in the containers of the present invention comprise a medicament and a propellant. Examples of possible propellants include, but are not limited to, a C₁₋₄ hydrofluoroalkane, e.g., 1,1,1,2-tetrafluoroethane and 1,1,1,2,3,3,3-n-heptafluoropropane, or a mixture thereof as propellant. Other propellants may be used including, for example, alkanes (e.g., butane and propane), along with CO₂ (e.g., liquid CO₂).

Other embodiments of oral inhalers (e.g., MDI's), inhaler components and drug formulations useful in the present invention are disclosed in the commonly assigned U.S. Pat. Nos. 4,364,923; 6,309,624; 4,335,121; 6,251,368; 5,676,929; 5,674,471; 5,290,815; 5,126,375; 5,225,445; 4,922,474; 5,674,472; 5,658,549; 5,270,305; 6,303,103; 6,309,624; 6,315,173; 6,170,717; 6,318,603; 6,238,647; 6,119,853; 6,315,112; 6,179,118; 6,149,892; 6,253,762; 6,131,566; and, 6,143,277.

With respect to intranasal use, the devices employed to administer medicament(s) may be fabricated according to techniques known in the art. Examples of embodiments for use in intranasal administration include, without limitation, those described in U.S. Pat. Nos. 4,771,769, 4,860,738, Des. 295,787, 3,949,939, 4,311,255, 4,830,224, 4,921,142, 5,284,132, 5,894,963, 6,173,868, 6,276,568, 6,364,166, Des 144,555, 4,344,573, 4,513,819, 6,211,054 and 5,301,846, and published U.S. Patent Applications 2002/0010428, 2002/0011530 A1, the disclosures of which are incorporated herein by reference in their entirety.

In various intranasal embodiments, the container may be present as a pressurized canister, or in the form of a bottle formed from, for example, polymers, glass, metals, or combinations thereof. In a number of particular embodiments, the therapeutically effective compound/medicament administered intranasally may be generally formulated as a liquid media, typically in water, and may contain various components such as stabilizers, preservatives, surfactants, emulsifiers, suspending agents, solvents, co-solvents, solubilizers, tonicity agents, scents and flavorings/taste maskers. The therapeutically effective material may be formulated as a suspension or a solution in the liquid media. Where the medicament is generally non-soluble in an aqueous media, a suspension formulation is usually employed.

In a particular embodiment of the invention, preservatives, generally antimicrobial preservatives, include, comprise or may be selected from the group consisting of: pharmaceutical grades of benzalkonium chloride, methylparaben, sodium benzoate, benzoic acid, phenyl ethyl alcohol, mixtures thereof, and the like. In another preferred embodiment of the invention, the surfactant is selected from the group consisting of Polysorbate 80 NF, polyoxyethylene 20 sorbitan monolaurate, polyoxyethylene (4) sorbitan monolaurate, polyoxyethylene 20 sorbitan monopalmitate, polyoxyethylene 20 sorbitan monostearate, polyoxyethylene (4) sorbitan monostearate, polyoxyethylene 20 sorbitan tristearate, polyoxyethylene (5) sorbitan monooleate, polyoxyethylene 20 sorbitan trioleate, polyoxyethylene 20 sorbitan monoisostearate, sorbitan monooleate, sorbitan monolaurate, sorbitan monopalmitate, sorbitan monostearate, sorbitan trilaurate, sorbitan trioleate, sorbitan tristearate, mixtures thereof and the like. In yet another preferred embodiment of the invention, the tonicity agent is selected from the group consisting of: dextrose, lactose, sodium chloride, mixtures thereof, and the like. In yet another preferred embodiment of the invention, the suspending agent is comprises or is selected from the group consisting of: microcrystalline cellulose, carboxymethyl sodium NF, polyacrylic acid, magnesium aluminum silicate, xantham gum, mixtures thereof and the like.

In addition encompassing administration of liquid media, intranasal devices may also administer medicament(s) via an aerosol formulation. In a particular embodiment, the aerosol formulation can include one or more propellants, such as those described herein.

In another aspect, the invention relates to a method of administering at least one medicament to a patient. The method comprises providing a system as defined herein; and activating the system to deliver a pharmaceutically effective amount of the at least one medicament to the patient. Such methods may be used in the treatment of and/or the prophylaxis of a respiratory condition. For the purposes of the invention, the term “respiratory condition” encompasses, without limitation, diseases and disorders associated with reversible airways obstruction such as asthma, chronic obstructive pulmonary diseases (COPD) (e.g. chronic and wheezy bronchitis, emphysema), respiratory tract infection and upper respiratory tract disease (e.g. rhinitis, such as allergic and seasonal rhinitis).

The systems of the invention may also encompass other embodiments. As an example, at least one additional tubular nozzle may be employed in the system. The at least one additional tubular nozzle may be fabricated and assembled as described herein. Non-limiting embodiments that encompass utilizing at least one additional tubular nozzle are as follows: (1) an inlet which splits into a plurality of inlets for the individual tubular nozzles and (2) an outlet of a single tubular nozzle splitting into multiple outlets.

The invention will now be described in greater detail with respect to the embodiments described in the following drawings. It should be appreciated that the drawings serve to illustrate the invention, and do not limit the scope of the invention as defined by the claims. In the drawings, like numbers refer to like elements throughout.

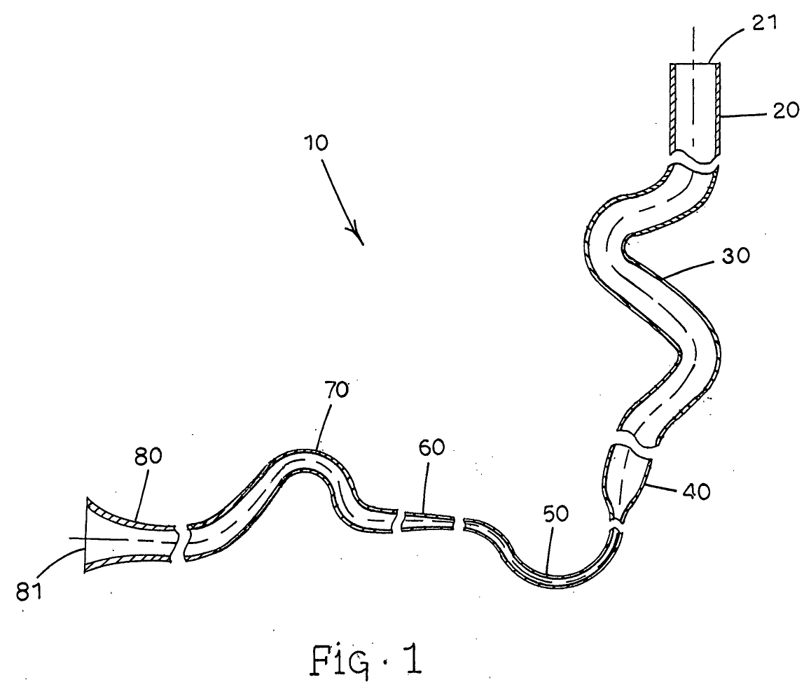

FIG. 1 illustrates an embodiment of a tubular nozzle 10 in cross sectional view according to the present invention. In this specific embodiment, the tubular nozzle has an inlet 20 with entrance 21 which is configured to sufficiently communicate with a dispensing passage positioned in a valve stem of a system such as an oral inhaler or an intranasal device. The inlet 20 may communicate with the dispensing passage in various manners. As an example, the inlet 20 can be configured for use in conjunction with a connector which engages the valve stem in a suitable manner.

A section 30 of the tubular nozzle is located proximal and adjacent to the nozzle inlet. As depicted in FIG. 1, section 30 has linear and non-linear (curved) portions. However, it should be appreciated by one skilled in the art that section 30 may be linear with no curved portion(s), or alternatively curved with no linear portions. Adjacent to section 30 is a tapered section 40. As illustrated in FIG. 1, the diameter of the tapered section 40 decreases in the direction moving downstream from the inlet of the tubular nozzle 10, i.e., decreases in the direction distal relative to the inlet. Alternatively, the tapered section could increase opposite from the inlet, in other words increase in the distal direction relative to the inlet.

Still referring to FIG. 1, section 50 continues from tapered section 40 and shifts the nozzle from a substantially vertical orientation to a substantially horizontal orientation. Accordingly, section 50 includes a curved portion. As the section of smallest inner diameter, section 50 is also the throat of the nozzle in the embodiment shown in FIG. 1. Adjacent to section 50 is a tapered section 60, which increases in diameter in the direction distal relative to the inlet. Section 70 of the tubular nozzle continues from tapered portion 60, and is depicted in FIG. 1 with linear and non-linear (curved) portions. As with section 30, section 70 may be linear with no curved portion(s), or curved with no linear portion(s).

An outlet 80 with exit 81 is present distal to segment 70 as depicted in FIG. 1. As shown, outlet 80 tapers (i.e., changes inner diameter) nonlinearly, increasing in the distal direction relative to the tubular nozzle inlet. Alternatively, outlet 80 could taper linearly, could have no taper, or could taper decreasing in the distal direction relative to the tubular nozzle inlet.

It should be emphasized that FIG. 1 merely represents one embodiment of the tubular nozzle according to the present invention, and that a sizeable number of variations can be made to this structure. As an example, the nozzle may contain linear segments in addition to, or in place of, curved portions, and may include various other tapered portions in addition to, or in place of, those set forth in FIG. 1. Further, the nozzle may exclude some or all of the linear, curved, and tapered sections between the inlet 20 and outlet 80 set forth in FIG. 1. In other variations, referring to FIG. 1, sections 30 or 70 could be sized to be the throat of the tubular nozzle instead of section 50, or sections 30 or 70 could be configured as the location of the transition from substantially horizontal to substantially vertical in place of section 50.

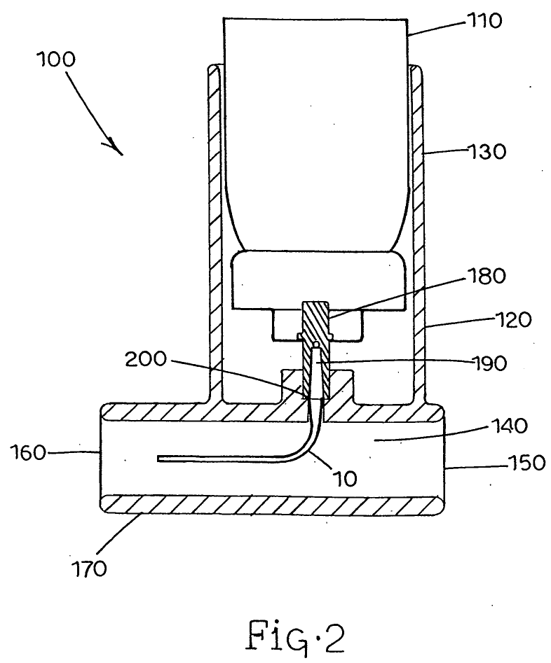

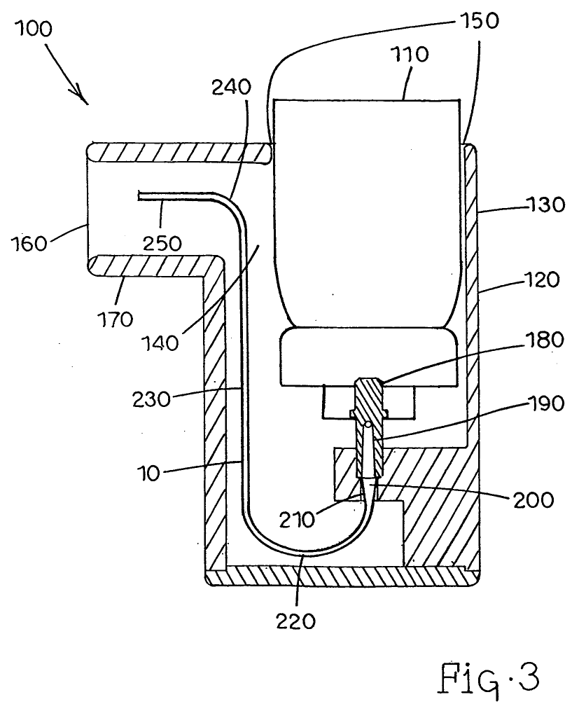

FIGS. 2 through 3 illustrate oral inhaler embodiments according to the present invention. It should be appreciated that variations from this embodiment may be made without departing from the scope of the invention.

FIG. 2 depicts an inhaler 100 (i.e., an MDI) which includes an aerosol canister 110. The MDI also includes an actuator 120 having a housing portion 130 for slidably engaging the canister 110 when the canister is actuated. Actuator 120 also includes an airflow passage 140 located at the bottom of the inhaler 100. The airflow passage 140 defines an air inlet 150 and an outlet 160 further defined by mouthpiece 170 to be engaged by an end user.

The aerosol canister 110 contains a pressurized pharmaceutical formulation as described herein. The formulation includes, without limitation, one or more medicaments and one or more propellants. Other ingredients may also be employed in the formulations such as, for example, excipients and additives referenced herein that include, without limitation, cosolvents, surfactants, flavorings, or other components. In this embodiment, the MDI also includes a metering valve assembly (not shown) as well as valve stem 180 and dispensing passage 190 positioned therein to allow a predetermined quantity of medicament to be delivered to a patient. Tubular nozzle 10 is present and extends substantially co-axially within the space created by the airflow passage 140. The inlet 200 of the tubular nozzle 10 is configured to communicate with the dispensing passage 190 within the valve stem 180 and advantageously directs a metered dose of medicament though the outlet of the mouthpiece 170 and into the lungs of a patient.

FIG. 3 depicts another embodiment of an inhaler 100 of the present invention employing a tubular nozzle. Similar to FIG. 2, the inhaler illustrated in FIG. 3 includes a canister 110, valve stem 180, dispensing passage 190, and tubular nozzle 10. Although not shown, a metering valve assembly may also be employed. In FIG. 3, a housing portion 130 of actuator 120 exists for slidably engaging the canister 110 when the canister is actuated, and additionally encloses the tubular nozzle 10. In this embodiment, the tubular nozzle 10 includes a tapered portion 210 at the proximal end of the nozzle relative to the dispensing passage 190, a first curved portion 220, a first linear portion 230, a second curved portion 240, and an outlet 250. Actuator 120 also includes an airflow passage 140 with air inlet 150 and outlet 160. As a result of this tubular nozzle configuration, outlet 160 is located at or substantially near the top of the inhaler 100, along with mouthpiece 170 for engagement by a patient.

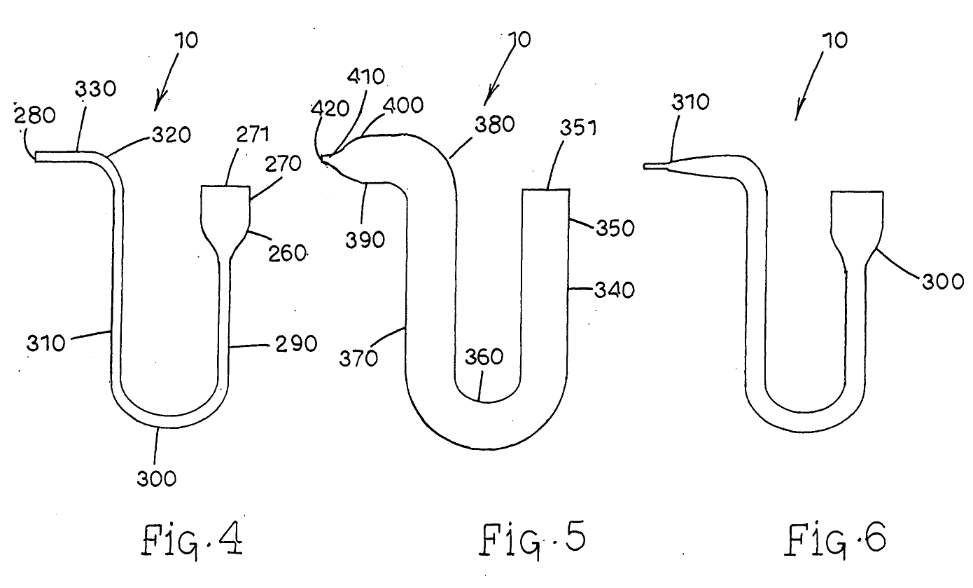

FIGS. 4 through 8 illustrate various configurations of tubular nozzles. It should be appreciated that other embodiments are encompassed by the invention in addition to those illustrated. Such embodiments may be employed in both oral and intranasal inhalation applications.

FIG. 4 illustrates a nozzle 10 having a tapered portion 260 proximal to the nozzle inlet 270, with entrance 271. The tapered portion 260 results in a reduction in nozzle diameter moving from nozzle inlet 270 to nozzle outlet 330. A first linear portion 290 extends downwardly from the tapered portion 260, followed by a first curved portion 300. Extending upwardly from curved portion 300 is a second linear portion 310. Second linear portion 310 is followed by a second curved portion 320. Extending from second curved portion 320 is nozzle outlet 330, terminating in exit 280.

FIG. 5 illustrates another embodiment of a tubular nozzle 10. In this embodiment, the nozzle 10 has a first linear portion 340 extending downwardly from the inlet 350 (with entrance 351) to first curved portion 360, i.e., the nozzle 10 does not contain a tapered portion immediately distal to the nozzle inlet. Second linear portion 370 extends upwardly from first curved portion 360. A second curved portion 380 follows from second linear portion 370, leading to straight portion 390 and tapered portion 400, ending in outlet 410 with exit 420. As depicted, tapered portion 400 is located substantially at the distal end of the nozzle and results in a decrease in nozzle diameter. In general, the internal volume of the nozzle illustrated in FIG. 5 is greater than the internal volume of that illustrated in FIG. 4.

FIG. 6 is similar to the nozzle of FIG. 4 in that a tapered portion 300 is present substantially near the proximal end of the nozzle 10. In this embodiment however, a second tapered portion 310 is present at the distal end of the nozzle, in a manner similar to FIG. 5. Thus, and as depicted, the nozzle undergoes two reductions in diameter.

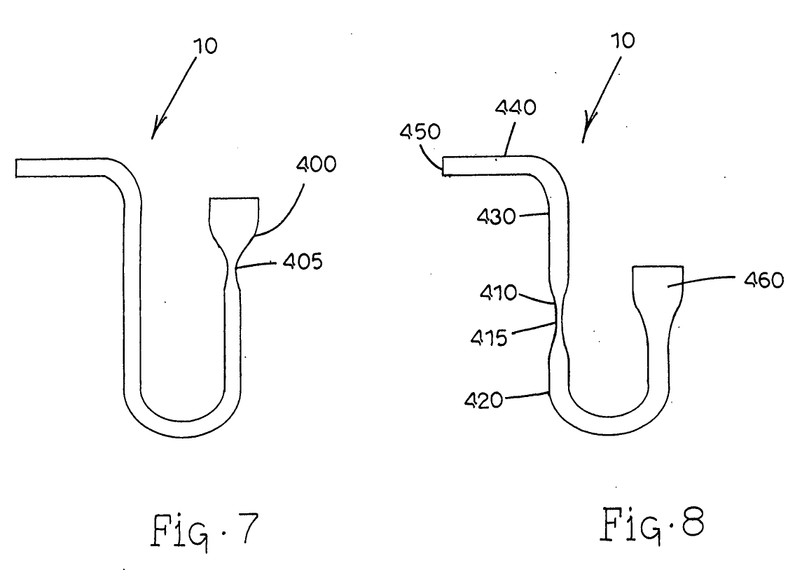

FIG. 7 illustrates a nozzle 10 similar to that set forth in FIG. 4. In FIG. 7, a tapered portion 400 is present proximal to the nozzle inlet. As shown, tapered section 400 results first in a decrease and subsequently an increase in nozzle diameter, creating the nozzle throat 405 at the portion of smallest diameter.

FIG. 8 illustrates a nozzle 10 having second tapered portion 410. As shown, portion 410 results in a sequential reduction and increase in tubular nozzle diameter, which creates the nozzle throat 415. Second and third linear portions, 420 and 430 respectively, precede and follow tapered portion 410. As illustrated in FIG. 8, outlet 440 and associated exit 450 are at heights above the tubular nozzle inlet 460.

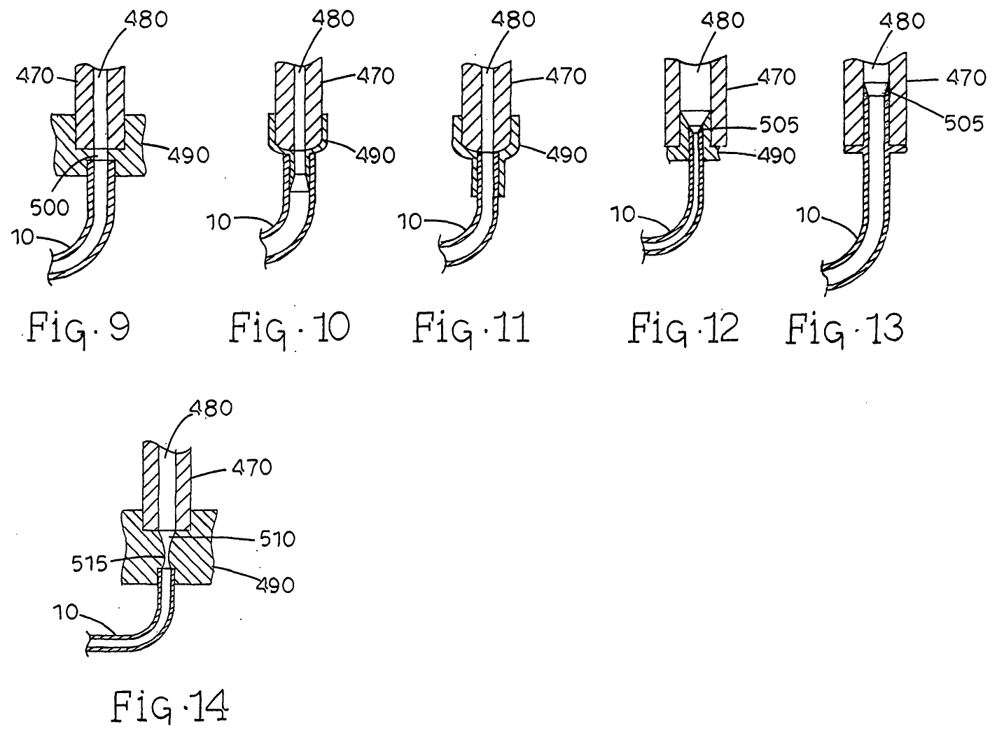

FIGS. 9 through 19 illustrate various embodiments depicting tubular nozzle inlets in communication with valve stem dispensing passages. Departures from these embodiments can be made without departing from the scope of the invention. In embodiments depicting a connector and tubular nozzle, such structures may be of one-piece or two-piece fabrication as set forth in detail herein. Such embodiments may be employed in both oral and intranasal inhalation applications.

FIG. 9 illustrates a tubular nozzle 10, a valve stem 470, and a dispensing passage 480 located therein positioned within a connector 490. In this embodiment, communication between the dispensing passage 480 and the tubular nozzle 10 is provided via an intermediate connecting channel 500. The connector 490 is depicted as being a finite square shape; nonetheless, it should be appreciated that this structure is very generally illustrated and may vary in configuration.

FIG. 10 illustrates a tubular nozzle 10 which connects to valve stem 470 via connector 490. In this embodiment, the tubular nozzle 10 has a larger inner diameter than the dispensing passage 480, and a tapered increase occurs within the connector 490.

FIG. 11 illustrates a tubular nozzle configuration 10 similar to that set forth in FIG. 9. In contrast to FIG. 9, the tubular nozzle 10 and the dispensing passage 480 are directly adjacent to one another.

FIG. 12 illustrates a tubular nozzle 10 which has a smaller inner diameter relative to the dispensing passage 480 within valve stem 470. In this embodiment, the taper affecting this change in diameter occurs within connector 490 and the tubular nozzle inlet 505.

FIG. 13 illustrates a tubular nozzle 10 which is configured to directly connect within valve stem 470 without employing a separate connector. As shown, the inner diameter of the nozzle is smaller relative to that of dispensing passage 480, and accordingly a tapered section 505 is present.

FIG. 14 illustrates a tubular nozzle 10 in communication with dispensing passage 480 utilizing connector 490. Similar to FIG. 9, such communication is provided via intermediate connecting channel 510. As shown in FIG. 14, connecting channel 510 results first in a decrease and subsequently an increase in diameter, creating the throat 515 at the portion of smallest diameter.

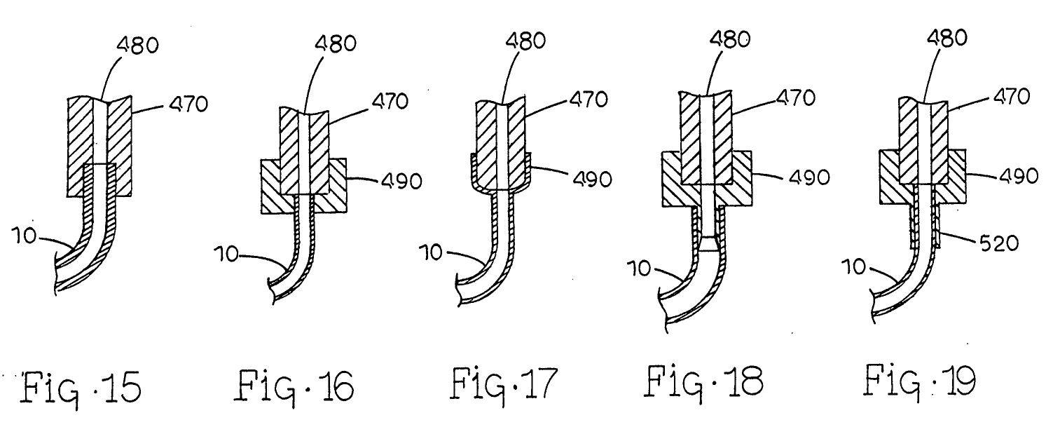

FIG. 15 illustrates a tubular nozzle that connects to valve stem 470 within dispensing passage 480.

FIG. 16 illustrates a tubular nozzle 10 in communication with dispensing passage 480 similar to the embodiment set forth in FIG. 11. In FIG. 16, the connector 490 is depicted as being a finite square shape; nonetheless, it should be appreciated that this structure is very generally illustrated and may vary in configuration.

FIG. 17 illustrates a tubular nozzle 10 directly connected to valve stem 470 via integral connector feature 490.

FIG. 18 illustrates a tubular nozzle 10 in communication with valve stem 470 similar to the embodiment set forth in FIG. 10. In FIG. 18 the connector 490 is depicted as a block, but could represent part of a larger structure, e.g., the actuator body.

FIG. 19 illustrates a tubular nozzle/valve stem configuration similar to the embodiment set forth in FIG. 16. In FIG. 19, additional nozzle support member 520 is present below connector 490 and extending co-axially along both sides of tubular nozzle 10.

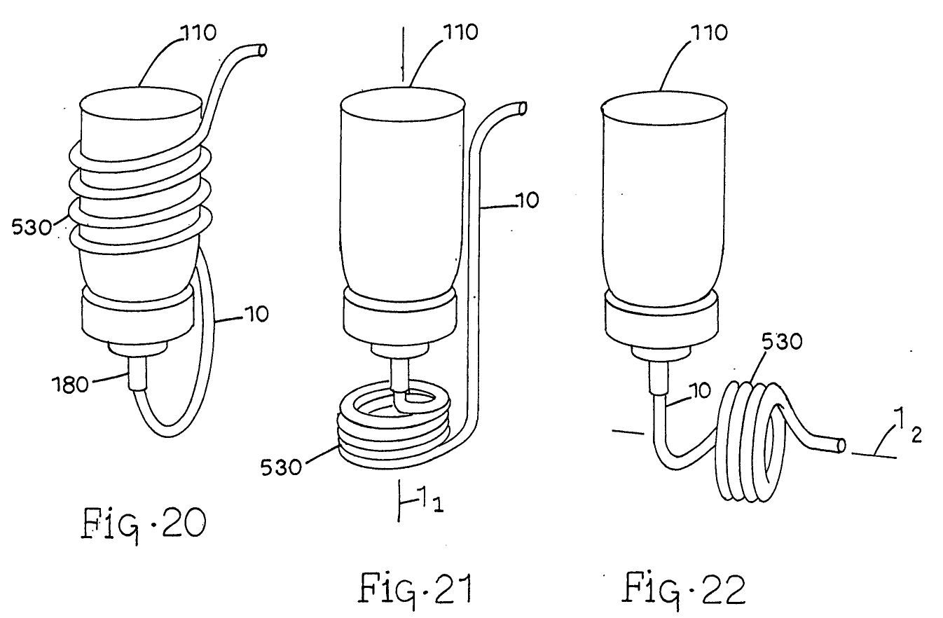

FIGS. 20 through 24 depict several embodiments in which a tubular nozzle is in communication with a canister and contains a coiled section which includes a plurality of coils. Notwithstanding these embodiments, it should be appreciated that other coiled configurations are within the scope of the present invention. For example, the coiled section may include only one coil or may include a portion of a full coil. Such embodiments may be employed in both oral and intranasal inhalation applications.

FIG. 20 depicts a tubular nozzle 10 which extends upward from valve stem 180 and includes coiled section 530 in which the coils surround the outer diameter of canister 110 such that each coil has a diameter substantially similar to the diameter of the canister 110.

FIG. 21 illustrates a tubular nozzle which contains a coiled section at a location below the bottom of canister 110. As shown in this embodiment, the coils are centered about an axis I₁ that defines the axis of the canister.

FIG. 22 illustrates a tubular nozzle 10 which has a coiled section 530 at a location below canister 110. As illustrated, the coils are centered around an axis I₂, which may be substantially the same as the central axis of the mouthpiece of the associated inhaler.

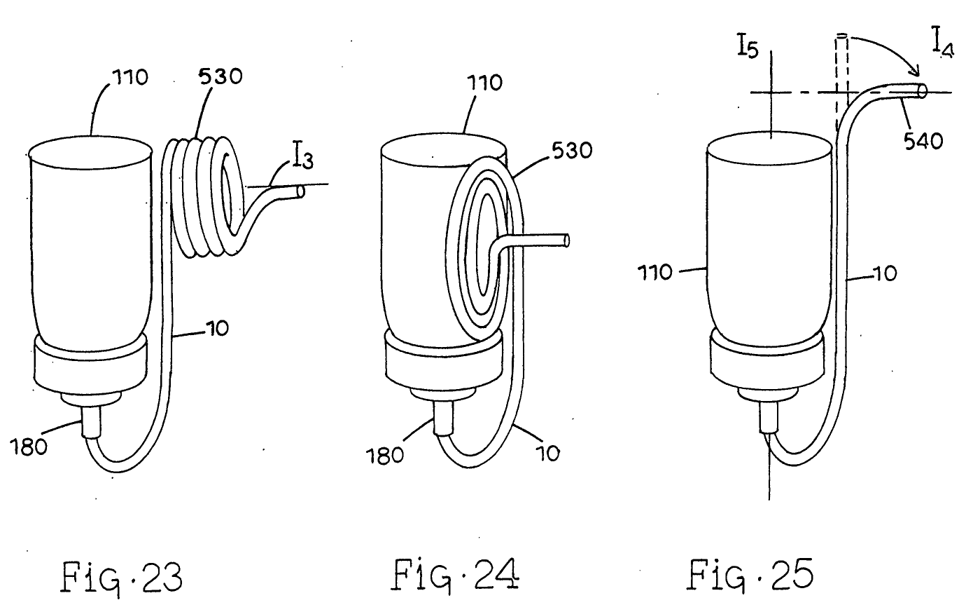

FIG. 23 depicts a tubular nozzle 10 which extends upward from the valve stem 180 and, at a location near the upper portion of the canister, is configured in a plurality of coils present as coiled section 530. As shown, the coils are centered about an axis I₃, which may be the same or substantially the same as the central axis of the mouthpiece of the associated inhaler.

FIG. 24 depicts a tubular nozzle 10 which extends upward from valve stem 180 and, at a location at or near the canister, is present in coiled section 530. As shown, the coils are concentrically present in substantially a single plane parallel to the axis that defines the length of canister 110.

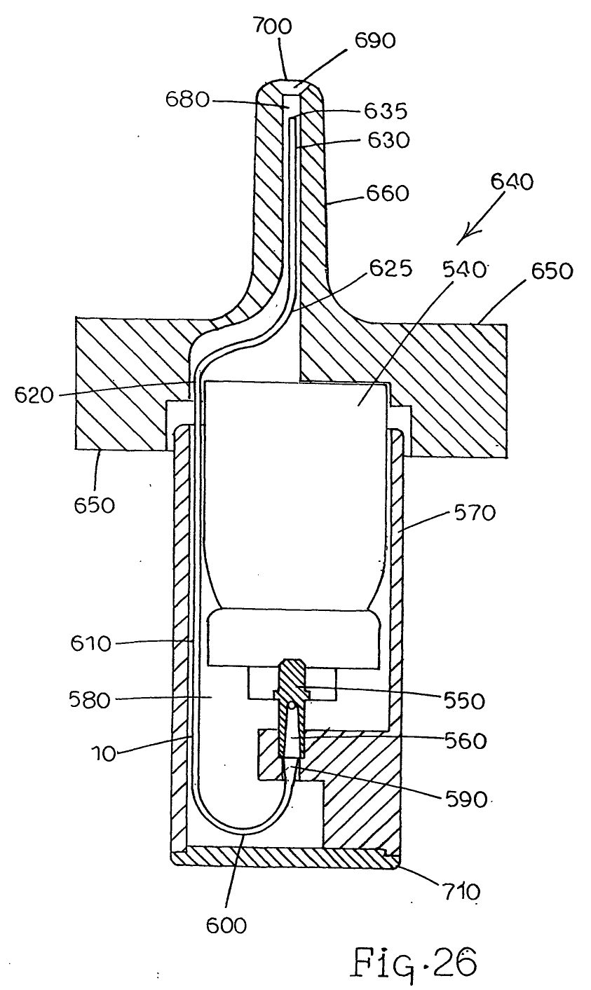

In addition to the above embodiments, the inhaler may be fabricated in a manner such that the tubular nozzle 10 has an outlet 540 which is substantially at or above the top of the canister, as depicted in FIG. 25. As shown by the arrow, the tubular nozzle may be flexible such that the outlet 540 can be moved from a position substantially aligned with canister axis I₅, to a position substantially perpendicular to axis I₅. Outlet 540 may be oriented in directions in addition to those set forth in FIG. 25.

An embodiment depicting an intranasal inhaler utilizing a tubular nozzle according to the present invention is set forth in FIG. 26. In particular, the device includes a canister 540 containing a pharmaceutical aerosol formulation, valve stem 550, dispensing passage 560, and tubular nozzle 10. Moreover, a housing 570 is present for slidably engaging the canister 540 when the canister is actuated, and opening 580 is present wherein tubular nozzle 10 resides. In this embodiment, the tubular nozzle 10 includes a tapered portion 590 at the proximal end of the nozzle relative to dispensing passage 560, a first curved portion 600, a first linear portion 610, second and third curved portions 620 and 625 respectively, a second linear portion 630, and an outlet 635.

Connected to housing 570 is an actuator 640. As shown, the actuator 640 includes outwardly projecting wings or finger grips 650 along with nasal adapter 660. Additionally, opening 680 is formed in head portion 640 and contains a portion of tubular nozzle 10 as shown. At its end portion, opening 680 contains a tapered section 690 that results in an increase in opening diameter near the exit orifice or nasal adapter outlet 700 of opening 680. In this embodiment, the end of tubular nozzle 10 is slightly recessed from exit orifice or nasal adapter outlet 700. Nonetheless, the tubular nozzle end may be even or substantially even with exit orifice or nasal adapter outlet 700, or may be recessed to an extent different from that illustrated in FIG. 26.

It should be appreciated that FIG. 26 merely represents one embodiment of an intranasal inhaler, and that variations from this embodiment may be encompassed within the scope of the present invention.

The intranasal inhaler may be operated according to accepted techniques. As an example, the intranasal inhaler is typically embraced by the patient on the outwardly projecting wings or finger grips and the inhaler bottom (denoted as 710). The inhaler is then actuated when the patient depresses the outwardly projecting wings or finger grips downward in relation to the inhaler bottom. The downward motion of the actuator causes the formulation to be drawn through the canister and ejected out the nozzle in the nasal adapter to the nasal cavity of the patient. It should be appreciated that the intranasal inhaler may be oriented at various angles relative to the nasal cavity of the patient.

With respect to oral and intranasal delivery systems, various embodiments may optionally be employed to assist in the atomization and/or aerosolization of the fluid dose of medicament. For example, in one embodiment, heat may be applied to the tubular nozzle in order to decrease the length required for the flow to develop into regimes with increased vapor fraction. Heat may be introduced in various manners including, without limitation, convection (e.g., by increasing the mass flow rate of air supplied to the nozzle), conduction (e.g., by providing a heat source, such as a resistance wire, in contact with the exterior of the tubular nozzle), or radiation (e.g., by irradiating the outer surface of the tubular nozzle with infrared radiation). In one embodiment, atomization may be assisted by vibrating the tubular nozzle during dose delivery such that the break-up of the fluid exiting the tubular nozzle outlet can be enhanced. The vibration may be introduced via numerous manners. For example, in one embodiment, the vibration may be longitudinal (i.e., in the direction of the nozzle outlet axis) or transverse (i.e., perpendicular to the nozzle outlet axis). A combination of longitudinal and transverse vibration can also be employed.

In general, the inhaler of the present invention employing the tubular nozzle is believed to be advantageous in reducing unwanted deposition in and on the inhaler device, in reducing unwanted deposition in the oropharynx, and in increasing the amount of medicament that reaches the lungs. It is believed that, by virtue of employing a tubular nozzle which is curvilinear without discontinuities, fluid flow from the canister may develop in a more regular, consistent manner relative to inhalers employing nozzles of irregular geometry (e.g., abrupt changes, dead flow areas, etc.). Further, it is believed that the absence of discontinuities and surfaces substantially perpendicular to the direction of flow prevents unwanted deposition of medicament particles, and their possible release in subsequent doses delivered by the inhaler. Finally, it is believed that influencing the vapor fraction of the dose of medicament/propellant formulation at the point of release from the nozzle exit influences the aerosolization of the medicament particles. In other words, the tubular nozzle of the invention permits potentially improved control of inhaled medicament delivery relative to conventional nozzles.

### EXAMPLES

The invention will now be further described by the examples that follow. It should be understood that such examples are for the purposes of illustrating the invention and are not intended to limit the scope of the invention.

Drug delivery performance of inhalers of the present invention having tubular nozzles of various dimensions were compared with a standard MDI, and with a standard MDI having an actuator modified for intended improved performance. Performance was determined by cascade impaction by collecting 10 shots from each inhaler fired into an Andersen cascade impactor (Thermo Andersen, Smyrna, Ga.) fitted with a modified USP (United States Pharmacopeia) throat at a flow rate of 28.3 L/min. The throat met the USP specification except that the length of the horizontal section of the throat was made to a length of 47 mm, rather than the standard 97 mm length. Drug deposition on the device, throat, and impactor were determined by high performance liquid chromotography (HPLC).

The throat deposition in the experimental apparatus is believed to be analogous to the oropharyngeal deposition in a patient, and the fine particle mass (FPM) is believed to be analogous to the deposition in the intended areas of drug delivery in the lung. Fine particle mass is defined here as the portion of the spray that is deposited on stages 3, 4, and 5 of the Anderson cascade impactor. Device deposition includes the nozzle, actuator, and valve stem.

All the inhalers compared used similar canisters and metering valves. The standard actuator is of conventional design and material. The modified standard actuator is injection molded like the standard actuator, but has a more smoothly contoured passage from the valve stem dispensing passage to the nozzle orifice and a smaller, shorter orifice.

### Examples 1-2

### Comparative Examples

Examples 1 and 2 represent conventional inhalers and have dimensions as set forth in Tables 1 and 2. Example 1 represents a standard actuator of conventional design and material and is injection molded. Example 2 represents a modified standard actuator which is injection molded similar to the actuator of Example 1, but has a more smoothly contoured passage from the valve stem to the orifice and a smaller, shorter orifice.

### Examples 3-6

### Actuators of the Invention

The actuators of the invention (Examples 3-6) have tubular nozzles with dimensions shown in Tables 1 and 2. The tubular nozzle of Example 3 is configured similar to that shown in FIG. 5, without the upward extending linear portion, and is made of stainless steel. The tubular nozzle of Example 6 is a longer version of the nozzle employed in Example 3. The tubular nozzles of Examples 4 and 5 are configured similar to that shown in FIG. 7, with the portion from the valve stem to the throat made of stainless steel, and the portion beyond the throat to the outlet made of PTFE polymer. Nozzles utilized in Examples 4 and 5 were of sufficient length to ensure that most or all liquid propellant was evaporated before exiting the nozzle outlet. The deposition figures in Tables 1 and 2 show the distribution of the drug particles emitted in terms of percentage of the total dose. Any deposition not accounted for in the tables was believed to have occurred on stages 0, 1, 2, 6, 7, or the filter placed after stage 7.

TABLE 1

| Actuator | Nozzle diameter | Nozzle length | Device | | |
|---|---|---|---|---|---|
| Example | (mm) | (mm) | deposition | Throat deposition | FPM |
| 1 | 0.50 | 1.5 | 20.5% | 53.0% | 18.5% |
| 3 | 0.25 | 24 | 20.1% | 28.3% | 43.7% |
| 4 | 0.50 | 416 | 22.2% | 31.2% | 34.1% |
| 5 | 0.25 | 511 | 27.0% | 14.3% | 48.5% |

TABLE 2

| Actuator | Nozzle diameter | Nozzle length | Device | | |
|---|---|---|---|---|---|
| Example | (mm) | (mm) | deposition | Throat deposition | FPM |
| 1 | 0.50 | 1.5 | 16.8% | 46.0% | 22.0% |
| 2 | 0.20 | 0.15 | 27.1% | 27.1% | 29.2% |
| 6 | 0.25 | 84 | 12.1% | 32.2% | 43.5% |

Table 1 illustrates comparable device deposition of the tubular nozzle actuators of the invention and the standard actuator. However, the tubular nozzle actuators of the present invention exhibit lower throat deposition and higher FPM than the standard actuator. Table 2 illustrates that modifying the conventional design reduces the throat deposition and increases the FPM, but device deposition is also significantly increased. An improvement in FPM and throat deposition is generally seen with the tubular nozzle devices of the invention.

Many unexpected results are demonstrated herein. For example, the degree of improvement in FPM is seen over a range of tubular nozzle dimensions compared to the standard actuator was unforeseen. Another surprising result is that using a tubular nozzle of such extended length as that in Examples 4 and 5 provides an increase in delivery efficiency with only a relatively small increase in device deposition. Also surprising is the performance improvement seen in Example 4, which has the same orifice diameter as the standard actuator of Example 1. Finally, these results show an unexpectedly higher margin of FPM improvement in general of the tubular nozzle actuators over the modified standard actuator of Example 2, when it is compared to the standard actuator of Example 1.

### Examples 7-21

### Tubular Nozzle Devices

Measurements and parameters of embodiments (i.e., Examples 7-18) according to the present invention and a control (i.e., a conventional nozzle) are shown in Table 3.

TABLE 3

| Nozzle | | | | | | | | Volume | Surface | SAᵢₙₜₑᵣₙₐₗ/ |
|---|---|---|---|---|---|---|---|---|---|---|
| Number | IDₚ, IDₗ | L_{d} | Lₗ + L_{c} | Lₗ | Lₜₒₜₐₗ | Lₜₒₜₐₗ/IDₚ | Lₜₒₜₐₗ/IDₗ | (V) | Areaᵢₙₜₑᵣₙₐₗ | V |
| 1 | (mm) | (mm) | (mm) | (mm) | (mm) | | | (μl) | (mm²) | ratio |
| 2 | 0.5, 1.4 | 3.2 | 12.1 | 5 | 24.3 | 48.6 | 17.4 | 10.0 | 51 | 5.1 |
| 3 | 0.5, 1.4 | 3.2 | 12.1 | 35 | 54.3 | 108.6 | 38.8 | 15.9 | 98 | 6.2 |
| 4 | 0.5, 1.4 | 3.2 | 12.1 | 65 | 84.3 | 168.6 | 60.2 | 21.8 | 145 | 6.7 |
| 5 | 0.25, 1.4 | 3.2 | 12.9 | 5 | 24.3 | 97.2 | 17.4 | 7.0 | 33 | 4.7 |
| 6 | 0.25, 1.4 | 3.2 | 12.9 | 35 | 54.3 | 217.2 | 38.8 | 8.5 | 57 | 6.7 |
| 7 | 0.25, 1.4 | 3.2 | 12.9 | 65 | 84.3 | 337.2 | 60.2 | 10.0 | 81 | 81 |
| 8 | 0.5, 1.4 | 3.2 | 15.5 | 5 | 24.3 | 48.6 | 17.4 | 33.2 | 101 | 3.0 |
| 9 | 0.5, 1.4 | 3.2 | 15.5 | 35 | 54.3 | 108.6 | 38.8 | 79.4 | 233 | 2.9 |
| 10 | 0.5, 1.4 | 3.2 | 15.5 | 65 | 84.3 | 168.6 | 60.2 | 125.6 | 365 | 2.9 |
| 11 | 0.25, 1.4 | 3.2 | 16.3 | 5 | 24.3 | 97.2 | 17.4 | 33.1 | 101 | 3.1 |
| 12 | 0.25, 1.4 | 3.2 | 16.3 | 35 | 54.3 | 217.2 | 38.8 | 79.3 | 233 | 2.9 |
| 13 | 0.25, 1.4 | 3.2 | 16.3 | 65 | 84.3 | 337.2 | 60.2 | 125.5 | 365 | 2.9 |
| 14 | 0.5, — | — | | — | 350 | | 700 | 68.7 | 550 | 8.0 |
| Control | 0.5, 1.4 | — | — | 1.5 | 1.5 | | 3 | 6.7 | 19 | 2.8 |

As detailed in the above table, nozzles 1 through 6 are Type A nozzles. Nozzles 7 through 12 are Type B nozzles evaluated using modified cascade impaction instrument for salmeterol xinafoate and fluticasone propionate.

The Dᵢₙₙₑᵣ is a measure of the inner diameter of the tubular nozzle in the non-tapered portion of the nozzle. The L_{straight} is a measure of the linear length of the straight portion of the tubular nozzle. The Lₜₒₜₐₗ is a measure of the total length of the tubular nozzle. The Lₜₒₜₐₗ/Dᵢₙₙₑᵣ is believed to be an important parameter of the present invention in that the ratio of the length of the tubular nozzle to the inner diameter is within some preferred range values such that at least some of the problems associated with conventional nozzles are overcome or substantially alleviated. The volume, V, is a measure of the internal volume of the tubular nozzle over the length Lₜₒₜₐₗ. The Surface Areaᵢₙₜₑᵣₙₐₗ, SAᵢₙₜₑᵣₙₐₗ, is a measure of the total internal surface area of the nozzle over the length Lₜₒₜₐₗ. The ratio SAᵢₙₜₑᵣₙₐₗ/V is also believed to be an important parameter of the present invention in that within some preferred ranges at least some of the problems associated with conventional nozzles are overcome or substantially alleviated.

### Example 22

### Drug Deposition Using Tubular Nozzles of the Invention

A drug formulation is prepared and filled into an MDI having a Model OF60 metering valve available from Valois of France. A conventional actuator was used to actuate the MDI. Nozzles 1-12 and the control were used with the same MDI and actuator. Using the different nozzles, the drug formulation was metered into a reduced Thermo Andersen Cascade Impactor available from ThermoAnderson of Bedford, Mass. The cascade impactor is an instrument commonly used to simulate the human lung to evaluate pulmonary drug delivery.

In this case, the filter stage represents drug delivery to the important lower/deeper regions of the lungs where drug is advantageously delivered. In contrast, it is believed to be disadvantageous to deliver drug to the throat because such drug is generally swallowed by the patient often causing undesirable side effects. Drug delivered to Stages 0, 1 and 2 are not nearly as important as delivering drug to the filter and reducing drug deposition to the nozzle and mouthpiece and reducing drug delivery to the throat. The analysis was conducted in accordance with U.S. Pharmacopia testing standards and guidelines.

The drug formulation was made by conventional mixing techniques at suitable temperatures and pressures for preparing aerosol drug formulations.

The drug formulation contained two drugs: micornised salmeterol xinafoate and micronised fluticasone propionate. No excipients were incorporated into the aerosol drug formulation. The strength of the dose was approximately 21 μg/actuation of salmeterol xinafoate and 220 μg/actuation of fluticasone propionate. The bulk formulation contqined approximately 0.0483% w/w salmeterol xinafoate, 0.3333% w/w fluticasone propionate, and 99.62% w/w P134a propellant (1,1,1,2-tetrafluoroethane). The amount in each actuation was approximately 36.25 μg salmeterol xinafoate (as salt), 250 μg fluticasone propionate and up to 75 mg propellant for the MOI having the control nozzle.

The drug formulation of Example 22 were evaluated by the reduced cascade impactor using the MDI having nozzles 1-12 and the control. The results for fluticasone propionate is shown in Table 4. The results for salmeterol xinafoate is shown in Table 5. The weight and identity of each drug on the nozzle, mouthpiece, total device, throat plate, stage 0, stage 1, stage 2 and filter plates were measured using HPLC (High Performance Liquid Chromatography). The cascade impactor was set to a flow rate of 28.3 liters/minute. The temperature, pressure and humidity were at ambient room conditions.

The weight data for the control on the nozzle and mouthpiece could not be individually measured because the conventional nozzles and mouthpieces are integrally moulded, i.e., they cannot be separated. However, the drug deposition on the total device was measured and is shown in the tables. The weights shown are the average weights for from 2-8 runs for each nozzle.

The drug formulations set forth in this example were evaluated for nozzles 1-12 and the control using the same MDI. The same modified cascade impaction instrument was used to measure drug accumulation./delivery. The results for fluticasone propionate is shown in Table 4, and the results for salmeterol xinafoate is shown in Table 5.

TABLE 4

| | | | Total | | | | | |
|---|---|---|---|---|---|---|---|---|
| | Nozzle | Mouthpiece | Device | Throat | | | | |
| Nozzle | Deposition | Deposition | Deposition | Deposition | Stage 0 | Stage 1 | Stage 2 | Filter |
| Number | (FP mcg.) | (FP, mcg.) | (FP, mcg.) | (FP, mcg.) | (FP, mcg.) | (FP, mcg.) | (FP, mcg.) | (FP, mcg.) |
| 1 | 7.0 | 1.3 | 8.3 | 20.3 | 3.3 | 3.8 | 5.9 | 58.6 |
| 2 | 10.3 | 1.3 | 11.6 | 20.5 | 4.1 | 5.2 | 6.4 | 52.3 |
| 3 | 4.4 | 0.9 | 5.2 | 22.1 | 5.7 | 7.4 | 8.6 | 51.2 |
| 4 | 4.4 | 2.9 | 7.3 | 35.5 | 4.5 | 5.7 | 6.8 | 40.4 |
| 5 | 4.1 | 4.8 | 8.9 | 42.2 | 2.9 | 4.0 | 4.6 | 37.5 |
| 6 | 3.9 | 7.8 | 11.7 | 32.8 | 4.6 | 4.4 | 5.1 | 41.6 |
| 7 | 4.6 | 2.9 | 7.5 | 26.4 | 2.4 | 3.1 | 4.8 | 56.1 |
| 8 | 8.7 | 3.4 | 12.0 | 30.6 | 2.1 | 2.7 | 4.4 | 48.2 |
| 9 | 19.9 | 1.7 | 21.6 | 23.5 | 1.9 | 1.1 | 2.7 | 49.4 |
| 10 | 2.7 | 7.4 | 10.1 | 39.9 | 2.7 | 4.0 | 4.9 | 38.6 |
| 11 | 6.6 | 3.6 | 10.1 | 34.3 | 5.6 | 5.2 | 4.9 | 40.1 |
| 12 | 6.6 | 8.8 | 15.3 | 35.8 | 3.5 | 3.6 | 4.4 | 37.4 |
| Control | n/a | n/a | 18.9 | 34.9 | 4.0 | 4.3 | 4.9 | 32.9 |

TABLE 5

| | | | Total | | | | | |
|---|---|---|---|---|---|---|---|---|
| | Nozzle | Mouthpiece | Device | Throat | | | | |
| Nozzle | Deposition | Deposition | Deposition | Deposition | Stage 0 | Stage 1 | Stage 2 | Filter |
| Number | (SX mcg.) | (SX, mcg.) | (SX, mcg.) | (SX, mcg.) | (SX, mcg.) | (SX, mcg.) | (SX, mcg.) | (SX, mcg.) |
| 1 | 4.3 | 3.2 | 7.4 | 18.1 | 1.8 | 1.8 | 2.7 | 68.2 |
| 2 | 9.4 | 1.2 | 10.6 | 20.0 | 4.3 | 5.7 | 7.2 | 52.3 |
| 3 | 4.0 | 0.8 | 4.8 | 21.9 | 5.8 | 7.8 | 9.2 | 50.6 |
| 4 | 4.2 | 2.9 | 7.1 | 34.4 | 4.8 | 6.3 | 7.5 | 40.1 |
| 5 | 3.8 | 4.7 | 8.5 | 42.4 | 3.1 | 4.4 | 5.1 | 38.6 |
| 6 | 3.6 | 7.6 | 11.2 | 32.3 | 4.7 | 4.7 | 5.5 | 41.7 |
| 7 | 4.3 | 2.5 | 6.7 | 24.1 | 2.3 | 3.5 | 5.4 | 58.1 |
| 8 | 7.8 | 2.9 | 10.7 | 27.3 | 2.3 | 3.0 | 5.0 | 51.9 |
| 9 | 18.2 | 1.3 | 19.5 | 19.9 | 1.7 | 1.3 | 3.1 | 54.6 |
| 10 | 2.6 | 6.8 | 9.4 | 38.8 | 2.9 | 4.3 | 5.4 | 39.3 |
| 11 | 6.4 | 3.6 | 10.0 | 35.2 | 5.8 | 5.5 | 5.3 | 38.5 |
| 12 | 7.2 | 5.0 | 12.2 | 39.7 | 2.1 | 1.4 | 2.4 | 42.4 |
| Control | n/a | n/a | 20.6 | 34.8 | 3.9 | 4.3 | 5.0 | 31.5 |

It is believed to be advantageous for as small a percentage as possible to accumulate in the nozzle and mouthpiece (i.e., the total device) so as to maximize the amount of emitted drug available to the patient. In contrast, it is believed to be advantageous to minimize the percentage of drug accumulating in the throat so as to minimize the possible risk and severity of side effect due to swallowing and digesting the drug. It is also believed to be advantageous to minimize the percentage of drug accumulating in stages 0, 1 and 2 because (for local treatment in the lungs) little if any therapeutic benefit is believed to be realized by delivering drug to those stages. Moreover, it is believed that little, if any, drug delivered to stages 0, 1 and 2 may not be available for delivery to the lower stages.

Lastly, for local treatment in the lungs to the patient, it is desirable that drug be delivered and accumulated in the lower stages of the cascade impaction instrument. In this example, the lower stages are depicted by the filter stage. Drug delivered and accumulated in the filter may be advantageous since respiratory conditions such as, for example, asthma and COPD are believed to be treated in the lower stages.

In the present invention, nozzles 1-12 are believed to be unexpectedly superior to conventional MDIs incorporating a conventional spray nozzle (i.e., the control). More particularly, generally for both drugs (i.e., salmeterol xinafoate and fluticasone propionate), a higher percentage of emitted drug is delivered/accumulated by nozzles 1-12 that the control to the filter. In addition, a majority of the nozzles delivered less drug to the throat than the control. Similarly, except for nozzle 9, had less drug accumulation on the device than the control. This is believed to be beneficial in that reduced deposition/accumulation of emitted drug on the device potentially allows for more drug to be available for delivery to the desired therapeutic stages of the respiratory tract.

The invention will now be expressed by the claims which follow. The embodiments presented hereinabove do not limit the scope of the invention but instead illustrate such scope.

That which is claimed:

1. A system for delivering a pharmaceutical formulation to a patient, said system comprising:
a container having a pharmaceutical formulation comprising at least one medicament present therein;
a metering assembly in communication with said container;
a tubular nozzle having an inlet configured in size to communicate with the metering assembly, and an outlet for directing the medicament to a patient, wherein the tubular nozzle has at least one curved portion;
wherein the tubular nozzle has a defined length and a longitudinal axis that is curvilinear throughout the defined length of the tubular nozzle, the tubular nozzle having a radius of curvature of at least 2.5 times the inner diameter of the tubular nozzle present within the curved portion.

2. The system according to claim 1, wherein the tubular nozzle includes at least one tapered section.

3. The system according to claim 2, wherein the at least one tapered section has an angle θ less than about 45°.

4. The system according to claim 2, wherein the at least one tapered section is positioned at a proximal end of the tubular nozzle.

5. The system according to claim 2, wherein the at least one tapered section is positioned at a distal end of the tubular nozzle.

6. The system according to claim 2, wherein the at least one tapered section decreases in the direction of the distal end of the tubular nozzle.

7. The system according to claim 2, wherein the at least one tapered section increases in the direction of the distal end.

8. The system according to claim 1, wherein the tubular nozzle includes at least one linear portion.

9. The system according to claim 8, wherein the tubular nozzle includes a plurality of linear portions located at a distal end of the tubular nozzle and a proximal end of the tubular nozzle.

10. The system according to claim 1, wherein the tubular nozzle includes at least one throat.

11. The system according to claim 1, wherein the tubular nozzle is constructed from a metallic material.

12. The system according to claim 11, wherein the metallic material comprises a metal selected from the group consisting of stainless steel, gold, nickel, brass, aluminum, titanium, tantalum, iron, and combinations thereof.

13. The system according to claim 1, wherein the tubular nozzle is constructed from a polymeric material.

14. The system according to claim 13, wherein the polymeric material is selected from the group consisting of polyethylene (PE), polypropylene (PP), polymethylmethylacrylate (PMMA), polyvinyl chloride (PVC), polyvinyidiene chloride (PVDC), polyvinyl fluoride (PVF), polyvinyldiene fluoride (PVDF), polychlorotrifluoroethylene (PCTFE), polytetrafluoroethylene (PTFE), fluorinated ethylene propylene (FEP), perfluroroalkoxy (PFA), polyamide (PA), polyethylene terephthalate (PET), polybutylene terephthalate (PBT), polyetherimide (PEI), polyamideimide (PAI), polyimide (PI), polysulfone (PS), polyarylsulfone (PAS) polyethersulfone (PES), polyphenylene sulfide (PPS), polyetheretherketone (PEEK), polydimethylsiloxane (PDMS) and polycarbonate (PC), combinations thereof, and blends thereof.

15. The system according to claim 1, wherein the medicament is selected from the group consisting of analgesics, anginal preparations, antiallergics, antiinfectives, antihistimines, anti-inflammatories, antitussives, diuretics, hormones, therapeutic proteins, peptides, medicaments for treating erectile dysfunction, and combinations thereof.

16. The system according to claim 1, wherein the at least one medicament is selected from the group consisting of fluticasone, beclomethasone, salmeterol, albuterol, ipratropium, salts thereof, esters thereof, solvates thereof, and combinations thereof.

17. The system according to claim 1, wherein the at least one medicament comprises albuterol sulfate.

18. The system according to claim 1, wherein the at least one medicament comprises salmeterol xinafoate and fluticasone propionate.

19. The system according to claim 1, wherein the at least one medicament comprises fluticasone propionate.

20. The system according to claim 1, wherein the at least one medicament comprises beclomethasone dipropionate.

21. The system according to claim 1, wherein the outlet of the tubular nozzle is oriented substantially horizontal.

22. The system according to claim 1, wherein the system is present as an oral inhaler.

23. The system according to claim 1, wherein the system is present as an intranasal inhaler.

24. The system according to claim 1, further comprising at least one additional tubular nozzle.

25. The system according to claim 1, wherein the system is present as an oral inhaler, and wherein said container is present as a canister, said pharmaceutical formulation is present as a pharmaceutical aerosol formulation comprising the at least one medicament and at least one propellant, said metering assembly is present as a metering valve assembly including a valve stem, wherein a passage for dispensing the at least one medicament is positioned in the valve stem; and wherein the inlet of said tubular nozzle is configured in size to communicate with the dispensing passage.

26. The system according to claim 25, wherein the inlet of the tubular nozzle is substantially aligned with the dispensing passage.

27. The system according to claim 25, wherein the tubular nozzle includes at least one tapered section.

28. The system according to claim 27, wherein the at least one tapered section has an angle θ less than about 45°.

29. The system according to claim 27, wherein the at least one tapered section is positioned at a proximal end of the tubular nozzle.

30. The system according to claim 27, wherein the at least one tapered section is positioned at a distal end of the tubular nozzle.

31. The system according to claim 27, wherein the at least one tapered section decreases in the direction of the distal end of the tubular nozzle.

32. The system according to claim 27, wherein the at least one tapered section increases in the direction of the distal end.

33. The system according to claim 25, wherein the tubular nozzle includes at least one linear portion.

34. The system according to claim 33, wherein the tubular nozzle includes a plurality of linear portions located at a distal end of the tubular nozzle and a proximal end of the tubular nozzle.

35. The system according to claim 25, wherein the tubular nozzle includes at least one throat.

36. The system according to claim 25, wherein the inhaler further comprises a means of actuation containing the canister, and wherein said means of actuation assists in delivering medicament to a patient.

37. The system according to claim 25, further comprising a mouthpiece having an outlet, wherein a portion of the tubular nozzle containing the tubular nozzle outlet is positioned in the mouthpiece.

38. The system according to claim 37, wherein the outlet of the tubular nozzle has an exit orifice, and wherein the exit orifice is substantially even with the outlet of the mouthpiece.

39. The system according to claim 37, wherein the exit orifice of the tubular nozzle is recessed from the outlet of the mouthpiece.

40. The system according to claim 37, wherein the portion of the tubular nozzle containing the tubular nozzle outlet is coaxial with the central axis of the mouthpiece.

41. The system according to claim 37, wherein the tubular nozzle outlet is at an angle ranging from about 0° to about 30° relative to the central axis of the mouthpiece.

42. The system according to claim 25, further comprising a connector that receives a proximal end of the tubular nozzle, and wherein the connector engages the valve stem.

43. The system according to claim 42, wherein the tubular nozzle and the connector are constructed from the same material.

44. The system according to claim 42, wherein the tubular nozzle and the connector are constructed from different material.

45. The system according to claim 25, wherein the tubular nozzle is constructed from a metallic material.

46. The system according to claim 45, wherein the metallic material comprises a metal selected from the group consisting of stainless steel, gold, nickel, brass, aluminum, titanium, tantalum, iron, and combinations thereof.

47. The system according to claim 25, wherein the tubular nozzle is constructed from a polymeric material.

48. The system according to claim 47, wherein the polymeric material is selected from the group consisting of polyethylene (PE), polypropylene (PP), polymethylmethylacrylate (PMMA), polyvinyl chloride (PVC), polyvinyidiene chloride (PVDC), polyvinyl fluoride (PVF), polyvinyldiene fluoride (PVDF), polychlorotrifluoroethylene (PCTFE), polytetrafluoroethylene (PTFE), fluorinated ethylene propylene (FEP), perfluroroalkoxy (PFA), polyamide (PA), polyethylene terephthalate (PET), polybutylene terephthalate (PBT), polyetherimide (PEI), polyamideimide (PAI), polyimide (PI), polysulfone (PS), polyarylsulfone (PAS) polyethersulfone (PES), polyphenylene sulfide (PPS), polyetheretherketone (PEEK), polydimethylsiloxane (PDMS) and polycarbonate (PC), combinations thereof, and blends thereof.

49. The system according to claim 25, wherein the diameter of the inlet of the tubular nozzle is similar to the diameter of the dispensing passage.

50. The system according to claim 25, wherein the medicament is selected from the group consisting of analgesics, anginal preparations, antiallergics, anti-infectives, antihistimines, anti-inflammatories, antitussives, diuretics, hormones, therapeutic proteins, peptides, medicaments for treating erectile dysfunction, and combinations thereof.

51. The system according to claim 25, wherein the at least one medicament is selected from the group consisting of fluticasone, beclomethasone, salmeterol, albuterol, ipratropium, salts thereof, esters thereof, solvates thereof, and combinations thereof.

52. The system according to claim 25, wherein the at least one medicament comprises albuterol sulfate.

53. The system according to claim 25, wherein the at least one medicament comprises salmeterol xinafoate and fluticasone propionate.

54. The system according to claim 25, wherein the at least one medicament comprises fluticasone propionate.

55. The system according to claim 25, wherein the at least one medicament comprises beclomethasone dipropionate.

56. The system according to claim 25, wherein the outlet of the tubular nozzle is oriented substantially horizontal.

57. The system according to claim 25, further comprising at least one additional tubular nozzle.

58. A method of administering at least one medicament to a patient, said method comprising:
providing a system as defined by claim 1; and
activating the system to deliver the at least one medicament to the patient.

59. The method according to claim 57, wherein the at least one medicament is selected from the group consisting of analgesics, anginal preparations, antiallergics, anti-infectives, antihistimines, anti-inflammatories, antitussives, diuretics, hormones, therapeutic proteins, peptides, medicaments for treating erectile dysfunction, and combinations thereof.

60. The method according to claim 57, wherein the at least one medicament is selected from the group consisting of fluticasone, beclomethasone, salmeterol, albuterol, ipratropium, salts thereof, esters thereof, solvates thereof, and combinations thereof.

61. The method according to claim 57, wherein the at least one medicament comprises albuterol sulfate.

62. The method according to claim 57, wherein the at least one medicament comprises salmeterol xinafoate and fluticasone propionate.

63. The method according to claim 57, wherein the at least one medicament comprises fluticasone propionate.

64. The method according to claim 57, wherein the at least one medicament comprises beclomethasone dipropionate.
